(19) United States
(12) Patent Application Publication
(10) Pub. No.: US 2026/0157888 A1
(43) Pub. Date: Jun. 11, 2026

(54) METHODS FOR USING VIOLET LASER ENERGY TO ADJUST THE REFRACTIVE PROPERTIES OF IMPLANTED INTRAOCULAR LENSES

(71) Applicant: Alcon Inc., Fribourg (CH)

(72) Inventor: Zsolt BOR, San Clemente, CA (US)

(21) Appl. No.: 19/413,483

(22) Filed: Dec. 9, 2025

Related U.S. Application Data
(60) Provisional application No. 63/730,909, filed on Dec. 11, 2024.

(51) Int. Cl. A61F 9/008; A61B 18/00; A61F 2/16

(52) U.S. Cl. CPC A61F 9/008; A61F 2/16; A61B 2018/00702; A61F 2002/16965; A61F 2009/00842; A61F 2009/0087

(57) ABSTRACT

Described herein is a method for adjusting at least one optical characteristic of an intraocular lens. The method involves the use of a violet laser having a wavelength in the range of 380 nm to 460 nm. The violet laser can be used to correct post-operative refractive errors in intraocular lenses, while being noninvasive and safe.

### CROSS-REFERENCE TO RELATED APPLICATIONS

This application claims the benefit of U.S. Provisional Patent Application No. 63/730,909, filed Dec. 11, 2024, which is incorporated by reference herein in its entirety, and is hereby expressly made a part of this specification.

### INTRODUCTION

Accurate ocular measurements are important for determining the correct power of an intraocular lens (IOL) to be implanted during cataract surgery. Optical biometry is a non-invasive method for measuring the optical and geometrical characteristics of the eye, and is the industry standard for pre-operative IOL power calculations. However, even advanced optical biometry measurements can lead to unpredictable or poor post-operative outcomes.

Further, postoperative refractive errors can also be caused by unpredictable patient specific healing processes. One out of five patients has a post-operative refractive error exceeding ±0.5 diopter. Refractive error exceeding ±0.5 diopter results in low visual acuity and reduced patient satisfaction.

### SUMMARY

Certain embodiments described herein are directed to methods and systems for adjusting refractive properties of an implanted IOL. Some embodiments include a method herein referred to as Violet Laser Adjustable Lens (VIOLAL) treatment. In some embodiments, VIOLAL treatment includes the use of a violet laser having a wavelength of from 380 nm to 460 nm. The violet laser can be used to correct postoperative refractive errors and to avoid IOL exchange surgery.

VIOLAL treatment can also be employed to adjust IOL properties as a patient's vision changes over time. VIOLAL treatment also can be used in contact lens manufacturing and individual patient specific contact lens treatment. VIOLAL treatment in accordance with the embodiments described herein can be repeated in case if the refraction of the patient changes with time, if the first adjustment was not sufficiently accurate, or if the patient is unsatisfied with the visual outcome.

Some embodiments of the present disclosure are directed to a method of modifying the spatial profile of the refractive index of at least a portion of an IOL. In some embodiments, the method comprises measuring a post-surgical refractive profile of a patient's eye. The method includes determining a corrective refractive profile for the implanted IOL based on the measured profile. Some embodiments include irradiating the implanted IOL with light from a laser having a wavelength in the violet spectral range of 380 nm to 460 nm.

In some embodiments, determining the corrective refractive profile further comprises identifying one or more locations within the IOL to be modified. In some embodiments, a method of modifying a refractive index of at least a portion of an IOL further comprises modifying a refractive index profile of an IOL, or a refractive index of at least a portion of a material of an IOL, to create a diffractive structure, a refractive structure, or a combination thereof.

In some embodiments, irradiating the implanted IOL further comprises irradiating at a spatial profile corresponding to the one or more locations determined to have warranted a modification. In some embodiments, irradiating the implanted IOL comprises inducing a refractive index change of up to 0.05.

In some embodiments, the IOL comprises at least one type of absorbing chromophore molecule that absorbs light in a wavelength in the violet spectrum. In some embodiments, the absorbing chromophore molecules are provided substantially homogeneously throughout the IOL. The concentration of the absorbing chromophore molecules in the IOL is selected so that the absorption coefficient at the violet laser wavelength is in the range of 10 inverse centimeter (cm⁻¹) to 200 cm⁻¹. Some embodiments include at least one type of absorbing chromophore molecule that absorbs light in a 380 nm to 460 nm wavelength range.

Some embodiments include a scanner capable of directing the violet laser spot to the correct x/y position on the IOL despite possible intra-treatment movement of the eye. In some embodiments, irradiating the IOL comprises scanning the beam of laser light at a scanning rate based on a transient heat shock caused by the beam of laser light. The transient heat shock comprises an absorption of heat having a first duration of time and a dissipation of heat having a second duration of time, the first duration of time being substantially the same as the second duration of time. In some embodiments, an irradiated IOL material is heated to a transient temperature ranging from 200° C. to 600° C.

### BRIEF DESCRIPTION OF THE DRAWINGS

FIG. 1 is a schematic illustration of a violet laser IOL adjustment system, in accordance with one or more embodiments.

FIG. 2 is a flow chart depicting a method of violet laser IOL adjustment, in accordance with one or more embodiments.

FIG. 3 is a schematic illustration of an exemplary IOL, in accordance with one or more embodiments.

FIG. 4 is a graphical representation of a phase profile for an IOL, in accordance with one or more embodiments.

FIG. 5A is a graphical representation of violet laser induced phase shift, in accordance with one or more embodiments.

FIG. 5B is a flow chart depicting a method for determining violet laser phase shift, in accordance with one or more embodiments.

FIG. 6 is a wavefront diagram for phase front correction, in accordance with one or more embodiments.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure can be partially or fully implemented using known components, only those portions of such known components that are necessary for an understanding of the present disclosure will be described, and detailed descriptions of other portions of such known components will be omitted so as not to obscure the disclosure.

As used herein, the singular form of “a”, “an”, and “the” include plural references unless the context clearly dictates otherwise. As used herein, the statement that two or more parts or components are “coupled” shall mean that the parts are joined or operate together either directly or indirectly (i.e., through one or more intermediate parts or components, so long as a link occurs). As used herein, “directly coupled” means that two elements are directly in contact with each other. As used herein, “fixedly coupled” or “fixed” means that two components are coupled so as to move as one while maintaining a constant orientation relative to each other. As used herein, “operatively coupled” means that two elements are coupled in such a way that the two elements function together. It is to be understood that two elements “operatively coupled” does not require a direct connection or a permanent connection between them. As utilized herein, “substantially” means that any difference is negligible, such that any difference is within an operating tolerance that is known to persons of ordinary skill in the art and provides for the desired performance and outcomes as described in the embodiments described herein. Descriptions of numerical ranges are endpoints inclusive.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the exemplary embodiments described herein, an embodiment showing a singular component should not be considered limiting; rather, the disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

IOL implantation following cataract surgery is a frequently performed operation that can vastly improve the quality of a patient's life. Approximately eighty thousand cataract surgeries are made daily all around the world. The implantation of a properly-selected IOL improves patient satisfaction levels and leads to pleasing visual outcomes. Some techniques for ocular evaluation and pre-operative IOL power calculation involve the use of ultrasound-based methods. In the last decade, optical biometry, another ophthalmic evaluation technique, has emerged and become the gold standard in ophthalmic evaluation.

Optical biometry is based on the principle of partial coherence interferometry and provides more accurate results than ultrasound-based techniques. The corneal curvature is typically measured with corneal topographers. Very recently, OCT (optical coherence tomography) is also used as optical biometry. Measurement of the Purkinje reflections may also assist the accuracy of optical biometry.

IOL calculators use measurements provided by optical biometry in order to calculate the appropriate IOL power. Despite the improved accuracy of optical biometers and sophisticated IOL calculators, refractive surprises often happen. Refractive surprises are predominantly caused by the uncontrollable individual postsurgical healing effects of the eye or unwanted IOL movement or rotation or surgical damage of the ciliary zonules.

Accordingly, some embodiments described herein are directed to adjusting refractive properties (i.e., changing the spherical power, the toricity, multifocality and high order aberrations) of surgically implanted IOLs by VIOLAL treatment. VIOLAL treatment of the embodiments herein includes non-invasive, in vivo correction of residual refractive errors of cataract surgery and avoids the need for invasive IOL exchange surgery. As described in detail below, the adjustment of IOL power may be performed a few months after all postsurgical healing processes have ended. VIOLAL treatment described below may also be employed to adjust IOL properties as a patient's vision changes over time. Some embodiments of VIOLAL treatment are applied to extraocular lenses such as contact lenses and/or spectacle lenses, which is described in further detail below.

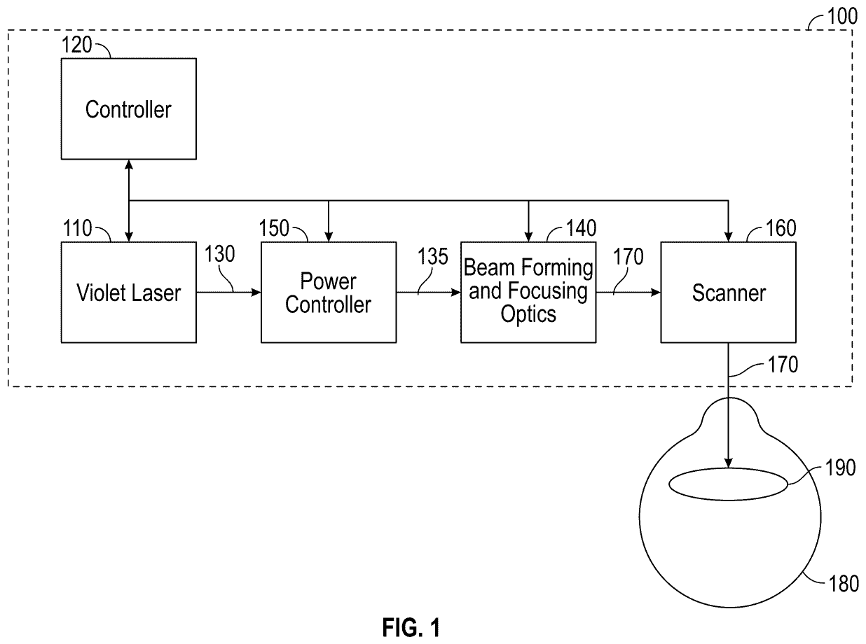

Referring now to FIG. 1, FIG. 1 depicts system 100 configured for VIOLAL treatment, in accordance with one or more embodiments described herein. In some embodiments, system 100 includes violet laser 110, controller 120, power controller 150, beam forming and focusing optics 140, and scanner 160. Controller 120 is communicatively coupled to components of system 100 (e.g., 110, 140, 150, 160) for sending and receiving commands/data and controls the operation of system 100 during VIOLAL treatment of IOL 190, which is discussed in further detail below.

As shown in FIG. 1, violet laser 110 emits violet laser beam 130. In some embodiments, violet laser 110 is a continuous wave laser, meaning that the power of violet laser beam 130 emitted by violet laser 110 is substantially constant during the time of treatment (i.e., not a pulse laser like the femtosecond laser that runs with 100 kHz to a few MHz repetition rate). For example, violet laser 110 may include a diode laser producing 380 nanometers (nm) to 460 nm violet light.

In some embodiments, power controller 150 may modify an intensity and/or power of violet laser beam 130 and output a modified beam 135 (e.g., of laser light) to beam forming and focusing optics 140. In some embodiments, power controller 150 may be an acousto-optical modulator. In some embodiments, when violet laser 110 is a diode laser, the power of modified beam 135 may be controlled electronically by regulating the current of the diode (not shown). In some embodiments, the power of modified beam 135 power is substantially in a range of 10 milliwatts (mW) to 2 Watts (W), or 10 mW to 500 mW.

In some embodiments, power controller 150 may cause the power of focused beam 170 (e.g., of laser light) to slowly vary during the treatment time. The treatment time (also referred to as exposure time), in some embodiments, may be limited to less than substantially 30 seconds to achieve the proper refractive index profile, as discussed in detail below. In some embodiments, the treatment time may be longer or shorter than 30 seconds.

In some embodiments, modified beam 135 may be focused to the appropriate diameter and/or spot size by beam forming and focusing optics 140, resulting in focused beam 170. In some embodiments, a spot size or spot size diameter for focused beam 170 may be substantially 40 micrometers (μm). In some embodiments, focused beam 170 includes a spot size having a diameter ranging between 1 μm to 100 μm, or between 10 μm to 100 μm. In some embodiments, focused beam 170 is substantially circular. In some embodiments, the intensity distribution of the laser spot can have a Gaussian, Super Gaussian, or flat top shape.

After emerging from beam forming and focusing optics 140, focused beam 170 is reflected by scanner 160. The angular deflection of focused beam 170 reflected from scanner 160 is also controlled by controller 120. Once reflected, focused beam 170 is then incident onto the proper x/y/z location of IOL 190 within patient's eye 180. In some embodiments, scanner 160 writes diffractive and/or refractive masks onto IOL 190 by irradiating focused beam 170 onto a portion of IOL 190. Focused beam 170 may induce transient heat shock at defined areas within IOL 190, which is described in detail below.

In some embodiments, scanner 160 may include a two-dimensional (2D) scanner. In such embodiments, scanner 160 may be integrated with an eye tracker (not shown) capable to direct the violet laser spot (e.g., focused beam 170) to the right x/y position of IOL 190, despite the intra-treatment movement of the eye. In some other embodiments, scanner 160 may include a three-dimensional (3D) scanner with an eye tracker. For any specific portion of IOL 190, the laser exposure time is equal to spot diameter of focused beam 170 divided by the scanning speed of scanner 160. In some embodiments, focused beam 170 is scanned at a speed ranging from 1 millimeter per second (mm/s) to 1,000 mm/s, or from 1 mm/s to 100 mm/s.

In some embodiments, focused beam 170 includes a wavelength substantially in the 380 nm to 460 nm violet spectral range with a 40 μm focused spot size. A 380 nm to 460 nm laser beam with 40 μm focused spot size has substantially a 4 mm Rayleigh length, which is much longer than the thickness of an IOL. The Rayleigh length of a laser beam refers to the distance along the propagation direction of the beam from the waist to the place where the area of the cross section is doubled. For a 380 nm to 460 nm laser, the longitudinal length of the refractive index change is not the Rayleigh length, rather, it is the absorption length 1/α of the violet laser light, where α is the absorption coefficient of IOL 190 (α=50 cm⁻¹). An absorption length of 1/50 cm⁻¹ corresponds to a longitudinal absorption length of 200 μm. With a 200 μm longitudinal interaction length, the required refractive index change to cause one wavelength (i.e., 0.5 μm) shift is 0.5 μm/200 μm=0.0025. In some embodiments, the spectral range of focused beam 170 may be extended to the 370 to 470 nm range.

In some embodiments, controller 120 manages the synchronized operation of violet laser 110, scanner 160, beam forming and focusing optics 140, and power controller 150 to generate focused beam 170 for achieving the proper spatial phase shift profile of IOL 190, which is discussed in detail below. The power of the laser irradiation is controlled by power controller 150 via commands from controller 120. In some embodiments, controller 120 is in communication with one or more ophthalmic biometry diagnostic tools and/or systems (not shown), discussed in further detail below.

Controller 120 includes a central processing unit (CPU), a memory, and support circuits. The CPU can be a general-purpose computer processor configured for use in an ophthalmic setting for controlling system 100. The memory can include random access memory, read-only memory, hard disk drive, or other suitable forms of digital storage, local or remote. The support circuits are conventionally coupled to the CPU and comprises cache, clock circuits, input/output subsystems, power supplied, and the like, and combinations thereof. Software routines, when executed by the CPU, transform the CPU into a specific purpose computer (controller) that controls system 100. Software routines (programs) and data can be coded and stored within the memory for instructing the processor within the CPU. A software program (or computer instructions) readable by CPU in controller 120 determines what tasks are performable by the components in system 100. The software routines can also be stored and/or executed by a second controller (not shown), such as a processing system controller, that is collocated with system 100.

In some embodiments, irradiating IOL 190 with laser light from focused beam 170 causes laser generated heat shock (discussed below), which causes at least one chemical reaction and/or physical change in IOL 190. The chemical reactions may include breaking of chemical bonds, partial depolymerization and formation of oligomers and monomers. The physical changes may include local polymer volume expansion, local polymer volume compaction, creation of empty spaces where smaller molecules can diffuse in or out, and/or diffusion of water molecules into an empty space of IOL 190. The refractive index of water is much less than the refractive index of IOL polymers. And the change of the water content in IOL 190 has the largest effect on the refractive index change. Therefore, diffusion of the water molecules may considerably change the refractive index of IOL 190.

One goal of VIOLAL treatment is to irradiate IOL 190 and cause a transient temperature elevation of the irradiated material of IOL 190 (i.e., transient heat shock) without causing material damage. The transient heat shock induced by VIOLAL treatment can change the refractive index of IOL 190, which is discussed in further detail below. The transient temperature elevation may, in some embodiments, be substantially in the range of 200 to substantially 600° C. Transient temperature elevations exceeding 600° C. may cause material damage to some IOLs.

Transient heat shock caused by laser irradiation of IOL 190 induces physical and chemical changes to the material of IOL 190. Laser-induced transient heat shock can cause breaking of IOL 190 physical and chemical bonds. Physical and chemical changes to materials of IOL 190 lead to a permanent change in the refractive index of IOL 190.

In some embodiments, heat caused by laser-irradiation can cause depolymerization. For example, depolymerization is a physical change where at least a portion of a polymer is broken down into oligomers and monomers. In some embodiments, laser-induced heat causes pyrolysis of IOL 190. In some embodiments, laser-induced heat causes local volumetric expansion of at least a portion of IOL 190. In some embodiments, laser-induced heat causes volume-compaction of IOL 190 polymers. In some embodiments, laser-induced heat leads to diffusion of molecules with small molecular weight out from a laser-irradiated volume. In some embodiments, laser-induced heat causes water molecules to diffuse into empty spaces within IOL 190 (i.e., the hygroscopy of the lend material is changing). Note that, “Laser-induced transient heat” and heat shock and “heat caused by laser-irradiation” and “laser-induced heat” are used interchangeably herein.

In some embodiments, IOL 190 may be configured to be optimized for absorbing violet light and thereby generating transient heat in IOL 190. In some embodiments, IOL 190 includes at least one type of photo-absorbing chromophore molecule that absorbs light in the 380 nm to 460 nm wavelength range. Chromophores embedded in IOL 190 are configured for single photon absorption of violet light, which optimizes laser-induced transient heat shock. IOL 190 may include one or more photo-activated chromophores and other materials. Other materials in IOL 190 may include collamer, hydrophobic acrylic, hydrophilic acrylic, PEG-PEA/HEMA/Styrene copolymer, polymethylmethacrylate (PMMA), silicone, and one or more chromophores.

Generally, IOL 190 may include polymers having a water content percentage by weight in a range between 0 wt % and 100 wt %. For example, in some embodiments, IOL 190 includes polymers having a water content percentage by weight in a range between 10 wt % and 90 wt %. In some embodiments, IOL 190 includes polymers having a water content percentage by weight in a range between 20 wt % and 80 wt %. In some embodiments, IOL 190 includes polymers having a water content percentage by weight in a range between 30 wt % and 70 wt %. In some embodiments, IOL 190 includes polymers having a water content percentage by weight in a range between 40 wt % and 60 wt %. In some embodiments, IOL 190 includes polymers having a water content percentage by weight greater than 50 wt %, greater than 60 wt %, greater than 70 wt %, greater than 80 wt %, or greater than 90 wt %. In some embodiments, IOL 190 includes polymers having a water content percentage by weight less than 50 wt %, less than 40 wt %, less than 30 wt %, less than 20 wt %, or less than 10 wt %.

The embodiments described herein may be analogously applied to extraocular lenses (EOLs), which are not surgically implanted into the eye, such as a contact lens or a spectacle lens worn externally by the patient. Similar to IOL 190, EOL materials include polymers containing 20%-80% water. EOL materials may also be doped with UV absorbing chromophores. Transient heat shock can also change the water content of BOL material. In this way VIOLAL method can be used in manufacturing of contact and/or spectacle lenses and to tailor the refractive power profile to the contact and/or spectacle lens to the individual needs of the patients. The treatment of EOL can happen in a manufacturing environment which is free of the ANSI laser eye safety regulations. The laser wavelength range can be extended into the 190 nm to 2 μm range. The treatment parameters such as laser power, scanning speed, laser spot size, chromophore concentration, chromophore absorption coefficient, treatment time can be optimized without considering the limitation applicable to the in vivo IOL treatment.

The embodiments of VIOLAL treatment are now further discussed in the context of Examples 1-3, infra. VIOLAL treatment allows for the modification or creation of at least one IOL characteristic (e.g., refractive power, refractive index, multifocality, toricity, and/or wave front aberrations). One or more embodiments described herein may change the refractive power of an IOL (e.g., IOL 190) by adding a diffractive structure (see Example 1, design of a +1 diopter diffractive Fresnel lens), refractive structure (see Example 2, design of a small area +2 diopter refractive lens in the implanted IOL to treat presbyopia), or a combination of diffractive and refractive structural components. As disused in further detail below, VIOLAL treatment may build any refractive index profile and may also correct high order aberrations (see Example 3, design of a wave front correcting treatment).

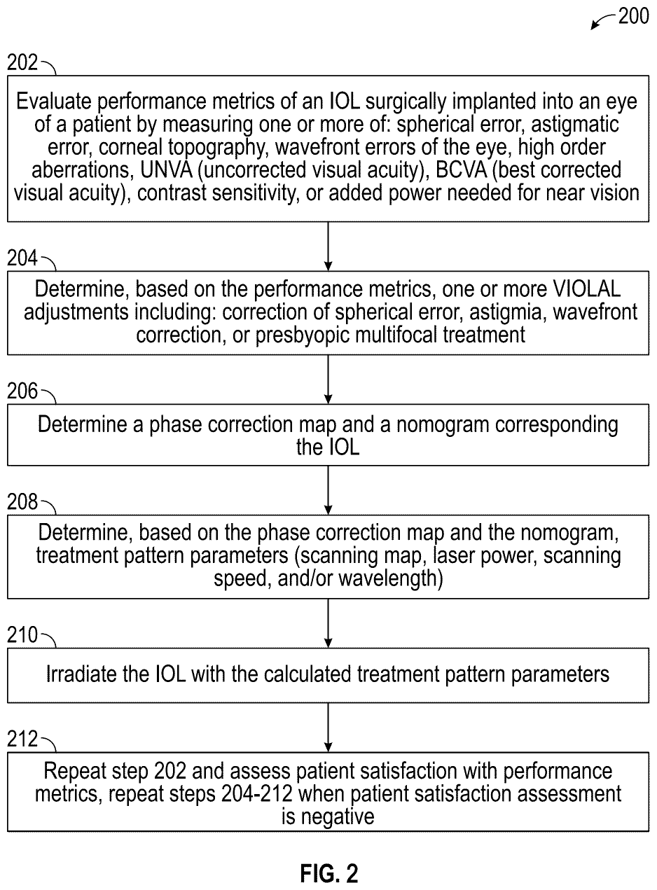

FIG. 2 illustrates a method 200 of VIOLAL treatment utilizing phase-shift nomograms. Method 200 is described below in conjunction with FIGS. 1 and 3-6 and may be implemented by a surgeon performing VIOLAL treatment on an ophthalmic patient. Method 200 involves the use of system 100, phase-shift nomograms (e.g., 500A), and/or ophthalmic biometry diagnostic tools. VIOLAL treatment, in some embodiments, starts after implanting the IOL into the patient's eye and waiting about 3 months until full healing of the eye. At step 202, the method begins by the surgeon evaluating performance metrics of an IOL that is surgically implanted into an eye of a patient by measuring one or more of: spherical error, astigmatic error, corneal topography, wavefront errors of the eye, high order aberrations, UNVA (uncorrected visual acuity), BCVA (best corrected visual acuity), contrast sensitivity, or added power needed for near vision. Such measurements may be taken utilizing ophthalmic biometry diagnostic tools in communication with system 100, discussed above. The surgeon consults the patient about the possible refractive correction including noninvasive VIOLAL treatment.

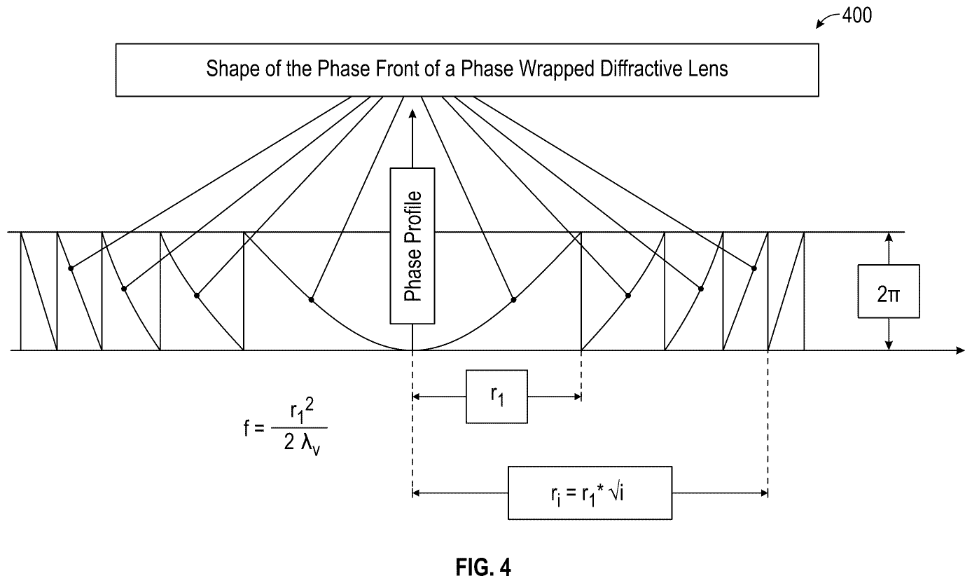

When the patient proceeds with VIOLAL treatment, the surgeon identifies one or more locations within the IOL to be modified, and, at step 204, determines, based on the locations and measured performance metrics, one or more VIOLAL adjustments including: correction of spherical error, astigmia, wavefront correction, and/or presbyopic multifocality treatment. At step 206, utilizing ophthalmic biometry diagnostic tools, the surgeon determines a phase correction map and a phase shift nomogram corresponding to IOL 190 (See e.g., FIGS. 4-6 and Examples 1-3, supra).

As mentioned below in Examples 1-3, in some embodiments, phase shift nomograms are derived experimentally by the surgeon utilizing ophthalmic biometry diagnostic tools (e.g., Hartmann-Shack Wavefront Sensor, Tscherning Aberrometer, Ray Tracing Aberrometry, OCT, and the like) for each patient at the time of VIOLAL treatment. In other embodiments, however, phase shift nomograms are cataloged by a technician in a lab and stored in a database accessible to system 100. For example, phase shift nomograms may be derived for all or many existing types of commonly used IOLs. In some embodiments, a database of lab-derived nomograms may be stored in a memory and/or a storage of controller 120. In some embodiments, the database of lab-derived nomograms may be stored remotely and accessible to system 100. Accordingly, in some embodiments, determining the phase shift nomogram may include inputting information about a specific type of IOL implanted in the patient's eye and retrieving stored nomograms corresponding to the specific type of IOL implanted in the patient from a database of IOL nomograms.

At step 208, the surgeon determines, based on the phase correction map and the phase shift nomogram, treatment pattern parameters (scanning map, laser power, scanning speed, and/or wavelength).

At step 210, the surgeon inputs treatment pattern parameters into system 100, which causes violet laser 110 to irradiate IOL 190 with the calculated treatment pattern parameters. Irradiating the IOL includes irradiating at a spatial profile corresponding to the one or more locations identified within the IOL to be modified. Irradiating the spatial profile may include utilizing a 3D scanner for irradiating several layers positioned above one another. For example, in some embodiments, irradiating the IOL includes irradiating, utilizing a 3D scanner (e.g., scanner 160), a first layer of IOL 190 and a second layer of the IOL 190, the second layer being below the first layer.

At step 212, repeat step 202 and assess patient satisfaction with performance metrics. The surgeon may assess the patient's satisfaction with IOL performance. For example, one week after VIOLAL treatment, the patient vision should be assessed again and a new VIOLAL treatment may be re-applied. When the patient assessment is positive (i.e., the patient is satisfied) the VIOLAL treatment method ends. When the patient assessment is negative (i.e., the patient is not satisfied), then steps 204-212 are repeated until the patient assessment is positive and/or desired performance metrics are produced.

### Example #1—Design of a Small Area +2 Diopter Refractive Lens

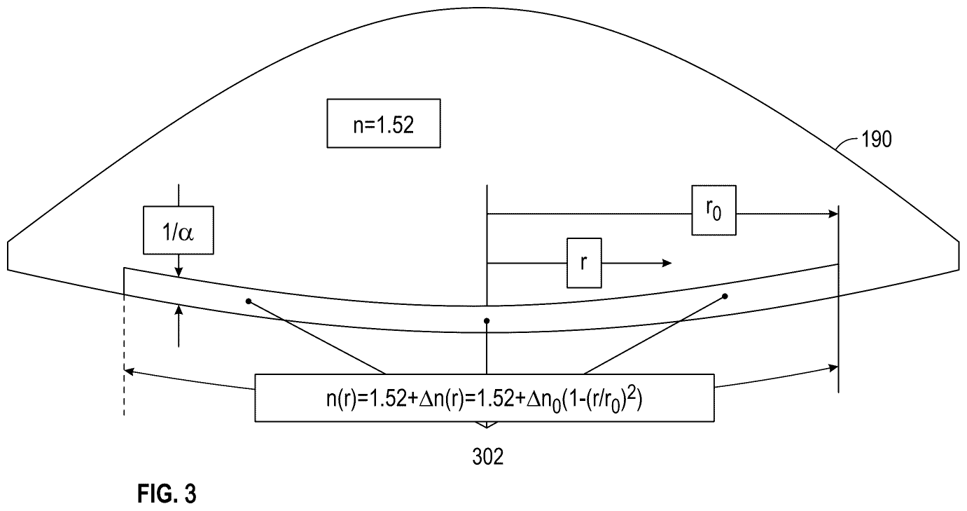

Referring now to FIG. 3, FIG. 3 depicts a schematic of IOL 190 having a refractive index, n, of 1.52, in accordance with some embodiments. One or more embodiments described herein may change the refractive power of IOL 190 by adding a refractive lens 302. IOL 190 may be configured for increasing the refractive power of the center of IOL 190 once implanted by f=+2.0 diopter. For example, as discussed below, the small area +2 diopter refractive lens 302 can be implanted IOL 190 to treat presbyopia. IOL 190 with refractive lens 302, in accordance with some embodiments, could be advantageous for an emmetropic and presbyopic person that uses laptop computers, smartphones, and reads books. The diameter of the added power may be 2r₀=2.0 mm. The surface area of a 2r₀=2.0 mm “added power” is derived by optometry to be enough to collect sufficient light flux for near reading.

In some embodiments, the penetration depth within the body of IOL 190 of violet laser 110 to form the +2 diopter refractive lens 302 may be 1/α= 1/50 cm⁻¹=200 μm. Optical calculations show that the optical path of the central beam may be increased by Δt with respect to an untreated IOL 190 where Δt is:

$$Δ t = \frac{{r}_{0}^{2}}{2 \cdot f} = \frac{{( 1. mm )}^{2}}{2 \cdot 500 mm} = 1. μ m \qquad ( 9 )$$

To have an optical path change of 1.0 μm over a distance of 1/α=200 μm, the required refractive index change Ano on the center (i.e., on the optical axis) is:

$$Δ {n}_{0} = \frac{1. μ m}{200 μ m} = 0.005 \qquad ( 10 )$$

For example, IOL 190 treated with femtosecond laser can have a Δn of up to 0.05, which is regarded to be the threshold of material damage. Thus Δn₀=0.005 needed for VIOLAL treatment, in accordance with some embodiments, is 10 times less than femtosecond laser methods. Therefore, by utilizing the embodiments described herein, there is no risk of damage in IOL 190.

In some embodiments, refractive lens 302 is configured for 2.0 added presbyopic spherical refraction. The radial profile of such refractive index change would be:

$$Δ n ( r ) = Δ {n}_{0} \cdot ( 1 - {( \frac{r}{{r}_{0}} )}^{2} ) \qquad ( 11 )$$

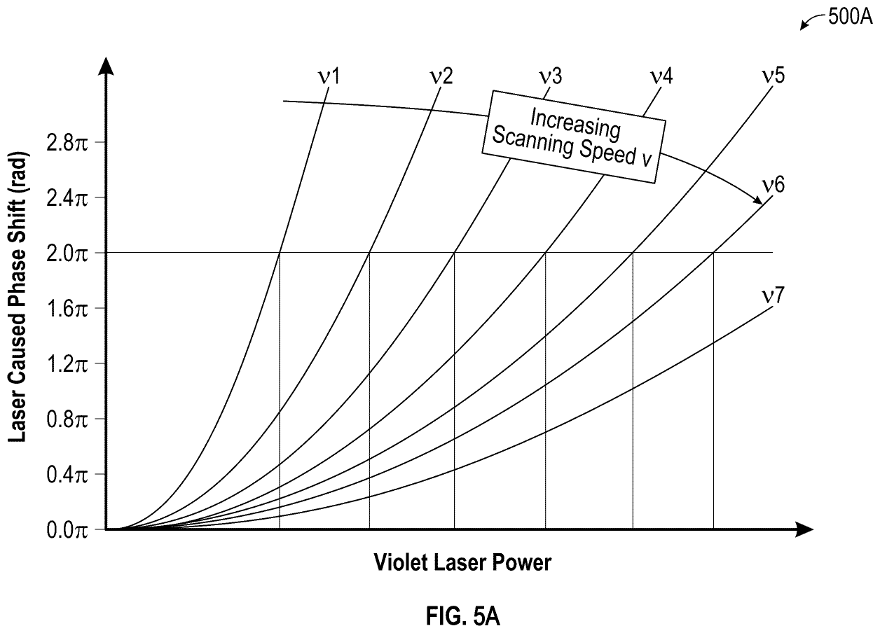

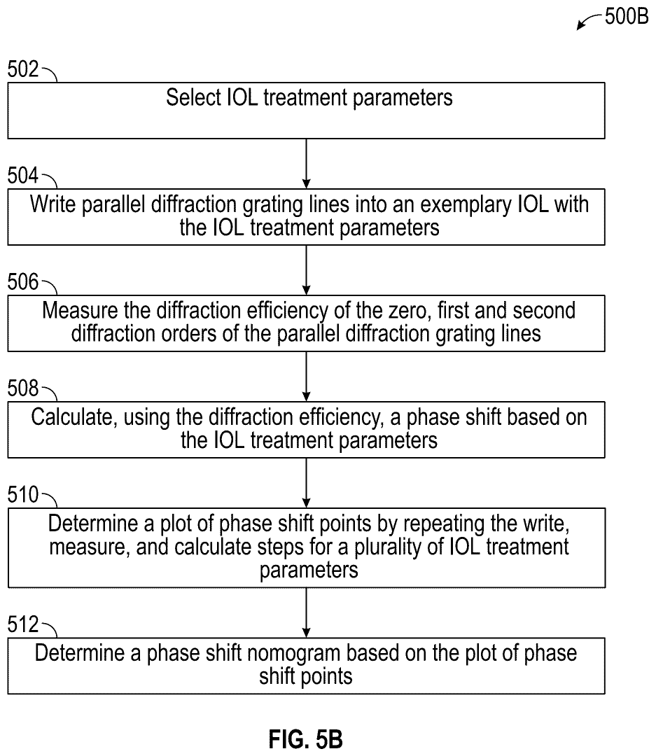

Such radial profile can be achieved using the power controller 150, with combination of nomogram 500A of FIG. 5A. When Δn deviates from Eqn. 11, then the added power will have unwanted spherical aberrations. Example 1 may be interpreted as a method to convert a monofocal IOL to bifocal. One or more embodiments described herein may change the refractive power of an IOL by correcting high order aberrations, for example, as shown in Example 3 below.

### Example 2—+1 Diopter Diffractive Lens

Referring now to FIGS. 4-6, in conjunction with FIG. 1, FIG. 4 shows the cross-section of the spatial profile 400 for the added phase shift of an exemplary diffractive lens, in accordance with some embodiments. Per theory of diffractive lenses, the shape of the central zone (Co) is a sphere having a focal length (f). FIG. 4 shows the cross-section thereof. The oblique lines are also the continuation of the same sphere, but interrupted with the 2π phase wrapping discontinuities. The purpose of the 2π phase jumps is to keep the span of the phase shift within the 0 to 2π range. To achieve the desired spatial phase shift profile, power controller 150 is used in combination with the nomogram shown on FIG. 5B, which is discussed further below.

As shown in FIG. 4, in some embodiments, for large phase correction, phase wrapping can be applied. Phase wrapping means multiple local jump-like phase shifts equal to + or −2π. 2π phase corresponds to an optical path difference equal to the wavelength of the visible light λᵥ. The purpose of phase wrapping is to keep the span of the phase variation within the range of 0 to 2π. The peak of the sensitivity of the human retina is at 550 nm therefore the λᵥ=550 nm may be selected.

In some embodiments, the maxima of the phase shift may be the 2π at the λᵥ=500 nm. The radii (rᵢ) of Fresnel rings are shown on FIG. 4. In many cases, post-surgical refractive error of a patient is a −1 diopter spherical error. In some embodiments, irradiating IOL 190 forms a +1 diopter diffractive Fresnel lens to compensate the −1 diopter post-surgical residual error.

The wavelength of the visible light for which the diffractive lens may be designed is λᵥ=500 nm. The diameter D of the optical zone of IOL 190 to be treated is D=4 mm. The power (P) of violet laser 110 may be selected to be P=0.084 W, which corresponds to the ANSI MPE (maximal permissible exposure) determined by the ANSI laser safety standards. The diameter of focused beam 170 incident onto IOL 190 may be selected to be Φ=40 μm.

In some embodiments, violet laser 110 wavelength may be λ=405. The absorption coefficient α of IOL 190 (at 405 nm wavelength of the violet laser) is α=50 cm⁻¹. The absorption coefficient depends on the concentration of the UV absorbing chromophore of IOL 190. Thus, α=50 cm⁻¹ is an exemplary value. In some embodiments, a concentration of the absorbing chromophore in IOL 190 is selected to have an absorption coefficient at the violet laser wavelength in the range of 10 cm⁻¹ to 200 cm⁻¹. The material properties of IOL 190 (e.g., density p, heat conductivity κ, specific heat c, and the like) are known and left out of the discussion for clarity.

Below, the output treatment parameters are calculated based on the input parameters. Output treatment parameters are the scanning speed (ν) of focused beam 170, the local transient exposure time (tᵣ), and the transient temperature elevation (ΔT) of an irradiated portion of IOL 190. The calculation below is an estimation of the output parameters, simplified for clarity. The sequence of the below calculation may aid in the understanding of the physics of VIOLAL treatment processes.

FIG. 5A shows the relationship between laser power and laser caused phase shift per scanning speed. As mentioned above, violet laser 110 is running with substantially continuous power. The temperature elevation from irradiating IOL 190 with focused beam 170 is caused by laser exposure. Thus, the rise time of the temperature (tᵣ) is equal to the local laser exposure time. At any given point, IOL 190 is exposed with the laser light for a duration equal to the diameter of the laser spot (Φ) divided by the scanning speed (ν). Thus, the local laser exposure time of a point of the IOL is:

$${t}_{r} = Φ / v$$

The cooling time, t_{cooling}, of a cylinder having a diameter may be calculated from the solution of the known heat conductivity equations using the numerical values of the material parameters (density ρ, heat conductivity κ, specific heat c) of an IOL:

$${t}_{cooling} = {( \frac{ϕ}{674 \frac{μ}{\sqrt{s}}} )}^{2} \qquad ( 2 )$$

### To Minimize the Retinal Laser Exposure, we Select:

$${t}_{r} = {t}_{c o o l i n g} \qquad ( 3 )$$

### Which is Used to Calculate the Scanning Speed as:

$$v = \frac{ϕ}{{t}_{r}} = \frac{ϕ}{{t}_{c o o l i n g}} = \frac{ϕ \cdot 67 {4}^{2} \frac{{μ}^{2}}{s}}{{ϕ}^{2}} = \frac{4.54 \times {10}^{5}}{ϕ} \frac{{μ}^{2}}{s} = \frac{4.54 \times {10}^{5}}{40 μ} \frac{{μ}^{2}}{s} = 1.135 \times 1 {0}^{4} \frac{μ m}{s} = 11.35 \frac{mm}{s} \qquad ( 4 )$$

When cooling time is substantially equal to rise time, it is advantageous for the physical changes and chemical reactions taking place in IOL 190 by increasing the accuracy of forming diffractive or refractive structures on IOL 190.

Some embodiments include estimating the transient temperature elevation. The energy density ε falling onto the Φ² surface of the IOL is approximately:

$$ϵ = \frac{P \cdot {t}_{r}}{{ϕ}^{2}} = \frac{P \cdot {t}_{c o o l i n g}}{{ϕ}^{2}} = \frac{P \cdot {ϕ}^{2}}{{ϕ}^{2} \cdot 67 {4}^{2}} \frac{s}{{μ}^{2}} = \frac{P}{4.54 \times {10}^{5}} \frac{s}{{μ}^{2}} \qquad ( 5 )$$

where P is the power of the violet laser.

### The Absorbed Laser Energy Causes a Transient Temperature Elevation of:

$$Δ T = \frac{ε \cdot α}{ρ \cdot c} = \frac{ε \cdot α}{1.13 \frac{g}{{cm}^{3}} \cdot 1.48 \frac{J}{g {\cdot }^{∘} C .}} = \frac{ε \cdot α}{1 \text{.76}} \frac{{∘}^{} C . \cdot {cm}^{3}}{J} \qquad ( 6 )$$

where α=50 cm⁻¹ is the absorption coefficient of the violet laser, ρ and c are the density and the specific heat of IOL 190.

### From Here:

$$Δ T = \frac{P \cdot α}{4.54 \times {10}^{5} \cdot 1.76} \frac{s}{{μ}^{2}} \frac{{∘}^{} C . \cdot {cm}^{3}}{J} = \frac{P \cdot α}{8 \times 1 {0}^{5}} {∘}^{} C . \frac{{cm}^{2}}{{μ}^{2}} \frac{cm \cdot s}{J} \qquad ( 7 )$$

$$Δ T = \frac{P \cdot α}{8} \times 1 {0}^{3} \cdot \frac{cm}{W} {∘}^{} C . = 125 \cdot P \cdot α \frac{cm}{W} {∘}^{} C . = 125 \cdot 0.084 W \cdot {cm}^{- 1} \frac{cm}{W} {∘}^{} C . = {525}^{∘} C .$$

Some embodiments include calculating the treatment procedure time (Tₚ). A Φ=40 μm laser spot scanned with a speed of ν=11.53 mm/s. The scanned laser beam treats a surface of νΦ in one second. Therefore, the treatment time of the optical zone of D=4 mm of the IOL is

$${T}_{p} = \frac{\frac{{D}^{2 \cdot π}}{4}}{υ \cdot ϕ} = \frac{{( 4 mm )}^{2} \cdot π}{4 \cdot 40 um \cdot 11.53 mm / s} = 27.6 s \qquad ( 8 )$$

The 27.6 second treatment time is a tolerable treatment time and does not challenge the stamina of the patient.

The VIOLAL treatment of the embodiments herein ensures a homogeneous refractive index change along the direction of the scanning. To have homogeneous refractive index change along the direction perpendicular to the scanning direction, there may be a partial overlap between the scanning tracks (not shown) of scanner 160. In some embodiments, the distance between the tracks may be smaller than the laser spot size on the IOL.

The distance between the scanning tracks and the degree of partial overlap affects the direction and amount of refractive index change. For example, wider track spacing results in less dense modifications, and, therefore, smaller refractive index change. Narrower scanning track spacing leads to a more dense pattern and a larger refractive index change

The degree of overlap between adjacent scanning tracks of scanner 160 influences the continuity of refractive index change patterns. A higher degree of overlap results in a more continuous pattern, providing uniform and predictable refractive index change. While a lower degree of overlap may result in less uniform patterns, and the potential for localized refractive index changes.

By adjusting the track spacing and partial overlap between scanning tracks of scanner 160, controller 120 may control the direction of the refractive index change. For example, in some embodiments, scanner 160 utilizes a radial pattern with a track spacing and overlap that result in a refractive index change that induces myopic or hyperopic corrections. In other embodiments, more complex refractive change patterns with different track spacing and overlap in different regions of the IOL may be implemented for addressing multifocality and/or astigmatism.

As shown above, with a scanning speed of 11.53 mm/s, the local laser exposure time (tᵣ) of a point of IOL 190 and the cooling time (t_{cooling}) are substantially equal (3.52 ms). This symmetry in heating and cooling time is advantageous because the amount of physical and chemical reactions are more predictable in this manner, which aids in creating the homogenous refractive index change in IOL 190. The 0.084 W violet laser power causes a 3.52 ms long 525° C. transient temperature elevation in a portion of IOL 190. 525° C. exceeds the glass temperature transition of the IOL polymer and also exceeds the supercritical temperature of the water and can cause thermochemical changes, therefore can cause large enough refractive index change. The 0.084 W ANSI MPE limited power of the laser is more than enough to cause large enough refractive index change. The power of violet laser 110 can be properly adjusted (e.g., using power controller 150) to achieve the required and planned refractive index and phase shift profile (e.g., FIG. 4).

Due to the transient nature of the heat shock, laser-induced phase shift cannot be calculated theoretically and, instead, it may be measured experimentally, which is discussed in further detail below. Experimental measurements should produce phase shift vs. violet laser power nomograms for different scanning speeds similar to what is shown in FIG. 5A. For the diffractive lens, a 500 nm optical path change may be achieved that causes a 2π phase shift. The absorption length of the violet light is 1/α= 1/50 cm⁻¹=200 μm. To have 2π phase shift over the distance of 200 μm the refractive index change approximately may be Δn=500 nm/200 μm=0.0025. A 0.0025 refractive index change does not cause catastrophic material damage of IOL 190.

FIG. 5B shows flowchart 500B depicting a method for determining phase shift in an exemplary IOL via a violet laser power phase shift nomogram. In some embodiments, the method for determining the phase shift nomogram may be performed by a technician in a lab environment. In other embodiments, phase shift may be determined by the surgeon at the time of VIOLAL treatment. At step 502, the method begins by the technician/surgeon selecting an IOL identical or similar to IOL 190 implanted into the eye of the patient. At step 504, the method continues by selecting arbitrary IOL treatment parameters (spot size, scanning speed, violet laser power, and wavelength). At step 506, the method continues by writing, utilizing violet laser 110, a parallel diffraction grating lines into the similar or identical IOL with one set of arbitrary treatment parameters.

At step 508, the method continues by measuring, utilizing ophthalmic biometry diagnostic tools, the diffraction efficiency of the zero, first and second diffraction orders. The diffraction efficiency may be measured in a lab environment or at the time of VOLAL treatment. At step 510, the method continues by using the results of the diffraction efficiency of the zero, first and second diffraction orders, apply the theory of diffraction for calculating the phase shift achieved with the selected treatment parameters. The calculated phase shift will give one point on the phase shift nomogram (e.g., FIG. 5A). At step 512, the method continuing by repeating the measurements with different sets of treatment parameters such that the quantity of measurement points will be sufficient to plot a treatment nomogram (e.g., similar to FIG. 5A).

### Example #3—Design of a Wave Front Correcting Treatment

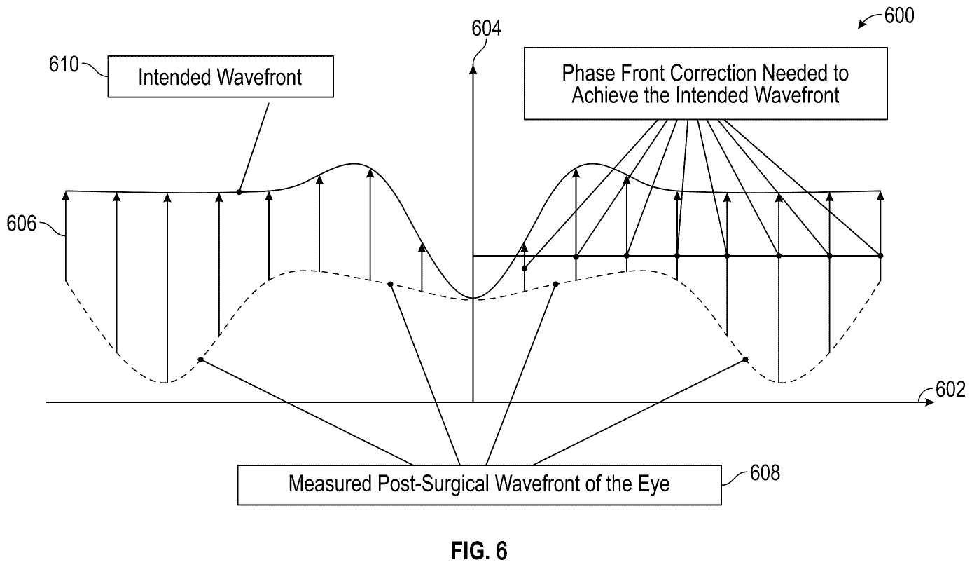

Referring now to FIG. 6, FIG. 6 shows a wavefront map 600 of a patient who is unsatisfied with the refractive outcome of an implanted IOL (e.g., IOL 190). Horizontal x-axis 602 is an imaginary line across the pupil of the patient; also shown is vertical y-axis 604. Ophthalmic biometry tools may measure and display wavefront map 600. For example, a wavefront meter (e.g., wavefront aberrometer) provides wavefront map 600 over the entire 2-dimensional x/y surface of the pupil. The change of the spatial phase profile results in the change of the refractive properties of IOL 190.

For example, in some embodiments, the surgeon may measure a post-surgical refractive wavefront 608 of patient eye 180 using and ophthalmic biometry diagnostic tools. For example, ophthalmic biometry diagnostic tools may include one or more of: Hartmann-Shack Wavefront Sensor, Tscherning Aberrometer, Ray Tracing Aberrometry, OCT, an optical bench laboratory, interferometry, image quality metrics, and/or computational modeling and simulation. The operation of ophthalmic biometry diagnostic tools are omitted herein for brevity. In some embodiments, ophthalmic biometry diagnostic tools are included within system 100. In some embodiments, ophthalmic biometry diagnostic tools are external to and in communication with system 100.

Utilizing ophthalmic biometry diagnostic tools, the surgeon may then determine a corrective refractive wavefront 610 for IOL 190 based on the measured post-surgical refractive wavefront 608. In some embodiments, determining the corrective refractive wavefront includes identifying, based on the wavefront map, one or more locations within IOL 190 to be modified. In some embodiments, the surgeon recommends the corrective refractive wavefront 610 to improve the visual acuity of the patient.

To convert the postsurgical refractive wavefront 608 to the corrective refractive wavefront 610, a phase correction treatment should be done with VIOLAL treatment of the embodiments described herein. During VIOLAL treatment, controller 120 controls the power of violet laser 110 and the x/y location of the laser spot of focused beam 170. Controller 120 controls the operation of system 100 for irradiating IOL 190 lens with light from violet laser 110 having a wavelength in the violet spectral range (i.e., 380 nm to 460 nm), at a spatial profile corresponding to the one or more locations determined to have warranted a modification to optimize wavefront properties. The magnitude of the correction is shown with vertical arrows 606. Using a nomogram similar to what is shown in FIG. 5A, software instructions (e.g., executed by controller 120) may calculate the power of the focused beam 170 as a function of the x/y position of focused beam 170 on IOL 190 behind the pupil of eye 180.

The above described embodiments may be implemented for converting monofocal IOLs to bifocal, multifocal, or extended depth of focus IOL. Conversely, bifocal, multifocal or extended depth of focus IOLs can be reversed to monofocal IOLs. The calculations presented above (i.e., Examples #1, #2 and #3) are simplified for clarity. A person having ordinary skill in the art would readily understand the design process and the orders of magnitude of the treatment parameters for implementing the operation of the embodiments described herein.

### Advantages

The embodiments described above enable adjusting refractive properties (i.e., changes in spherical power, change in toricity, increasing or reducing multifocality, and/or adjusting high order aberrations) of surgically implanted IOLs in a non-invasive manner after all postsurgical healing processes have ended. VIOLAL treatment is cost effective and avoids the need for invasive IOL exchange surgery. The embodiments described above may also be employed to adjust IOL properties as a patient's vision changes over time thereby increasing the lifespan of the IOL and increasing overall patient satisfaction with surgically implanted IOLs.

As described above, violet laser described herein may include a diode laser producing 380 nm to 460 nm violet light, which is a more cost effective solution in comparison to other types of lasers that can be used for the purposes described herein. Further, diode lasers are also more reliable, user-friendly devices in comparison with certain other lasers.

Further, as discussed above, the phase shift caused by VIOLAL treatment of the embodiments described herein is a linear, single photon process, and therefore is less sensitive to the treatment parameters of the laser in comparison to other existing solutions. Further, as described above, the violet laser described herein may generate a violet laser beam that is then focused with a wavelength substantially in the 380 nm to 460 nm violet spectral range with a 40 μm focused spot size. As also described above, the 380 nm to 460 nm laser may have a 200 μm longitudinal interaction length. With a 200 μm longitudinal interaction length, the required refractive index change to cause one wavelength (i.e., 0.5 □m) shift is 0.5 μm/200 μm=0.0025, which is 0.05/0.0025=20 times less than required for femtosecond laser treatment.

Also, an advantage of the large spot size of the violet laser beams used for the treatments described herein (e.g., focused beam 170) is that using a large spot size mitigates the need for immobilizing the eye of the patient. For example, because the scanner (e.g., scanner 160) that reflects the focused laser beams may include an eye tracker (not shown), which routinely may have 40 μm tracking accuracy, VIOLAL treatment does not require immobilizing the eye. Therefore, the VIOLAL treatment of the embodiments herein helps avoid the formation of corneal wrinkles during treatment, which may be caused by the use of patient interfaces for immobilizing the eye during treatment. In other words, because VIOLAL treatment does not involve the use of patient interfaces, corneal wrinkles can be avoided.

In addition, in comparison to smaller wavelength light treatments (e.g., UV light treatment) where no noticeable temperature change occurs in an IOL, according to some embodiments described herein, VIOLAL treatment may advantageously cause 200-600° C. transient heat shock in an IOL (e.g., IOL 190) for a few milliseconds, which triggers a refractive index/refractive power change in the IOL. Further, advantageously, the retinal laser exposure time of the embodiments described herein complies with the ANSI laser safety regulations for treatment/laser exposure time. The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments may fall within the scope of the appended claims.

What is claimed is:

1. A method of modifying a refractive index of at least a portion of an intraocular lens (IOL) for non-invasive, in vivo correction of one or more residual refractive errors, comprising:
measuring a refractive profile of a patient's eye;
determining a corrective refractive profile for the IOL based on the refractive profile; and
irradiating the IOL with a beam of laser light having a wavelength in a range of 380 nanometers (nm) to 460 nm.

2. The method of claim 1, wherein irradiating the IOL comprises scanning the beam of laser light at a scanning rate based on a transient heat shock caused by the beam of laser light.

3. The method of claim 2, wherein the transient heat shock comprises an absorption of heat having a first duration of time and a dissipation of heat having a second duration of time, the first duration of time being substantially equal to or less than the second duration of time.

4. The method of claim 1, wherein determining the corrective refractive profile further comprises identifying one or more locations within the IOL to be modified.

5. The method of claim 4, wherein irradiating the IOL further comprises irradiating at a spatial profile corresponding to the one or more locations.

6. The method of claim 1, wherein the beam of laser light incident on the IOL is scanned at a speed ranging from 1 millimeter per second (mm/s) to 1,000 mm/s.

7. The method of claim 1, wherein the beam of laser light corresponds to a continuous wave laser.

8. The method of claim 1, wherein the beam of laser light has a spot size diameter ranging from 1 micrometer (μm) to 100 μm.

9. The method of claim 1, wherein the beam of laser light has a laser power of 10 milliwatts (mW) to 2 Watts (W).

10. The method of claim 1, wherein an irradiated IOL material is heated to a transient temperature ranging from 200 degrees Celsius (° C.) to 600° C.

11. The method of claim 1, wherein a laser generated transient heat shock causes at least one chemical reaction in the IOL including a chemical bond break, monomer formation, oligomer formation, or depolymerization.

12. The method of claim 1, wherein a laser generated transient heat shock causes at least one physical change including: local volume expansion of the irradiated IOL, local polymer volume compaction, creation of empty space to be filled with molecules from a lens volume not irradiated by laser light, diffusion of water molecules into an empty spaces, or change a hygroscopy of a portion of the IOL exposed to the beam of laser light.

13. The method of claim 1, wherein one or more chromophores embedded in the IOL are configured for single photon absorption of violet light.

14. The method of claim 1, wherein the method further comprises modifying a spatial profile of the refractive index of the IOL to form a refractive lens in the IOL.

15. The method of claim 1, wherein the method further comprises modifying a spatial profile of the refractive index of the IOL to form a diffractive lens in the IOL.

16. The method of claim 1, wherein the method further comprises modifying a spatial profile of the refractive index of to optimize wavefront properties of the IOL.

17. The method of claim 1, wherein the method further comprises modifying a spatial profile of the refractive index of the IOL to combine one or more of a diffractive, a refractive and/or a wavefront modifying structure of the IOL.

18. The method of claim 1, wherein the method further comprises inducing a refractive index change of up to 0.05.

19. The method of claim 1, wherein the IOL comprises at least one type of absorbing chromophore molecule that absorbs light in a 380 nm to 460 nm wavelength range.

20. The method of claim 1, wherein the IOL comprises at least one type of absorbing chromophore molecule having an absorption coefficient at a violet laser wavelength in a range of 10 inverse centimeter (cm⁻¹) to 200 cm⁻¹.

21. The method of claim 1, wherein irradiating the IOL includes utilizing a 2D scanner integrated with an eye tracker capable of directing the beam of laser light to an accurate x/y position on the IOL despite intra-treatment movement of the patient's eye.

22. The method of claim 1, wherein irradiating the IOL includes irradiating, utilizing a 3D scanner, a first layer of the IOL and a second layer of the IOL, the second layer being below the first layer.

23. The method of claim 1, wherein the method is applied to modifying a refractive index of at least a portion of an extraocular lens worn external to the patient's eye.

24. The method of claim 23, wherein the method is applied to modifying a refractive index of at least a portion of a contact lens.

25. The method of claim 23, wherein the method is applied to modifying a refractive index of at least a portion of a material of a spectacle lens.
